SEQUENCE LISTING (3 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 275 aa, Bacillus amyloliquefaciens [sequence omitted]

SEQ ID NO: 3 - protein, 311 aa, Bacillus sp. [sequence omitted]

(19) United States
(12) Patent Application Publication
(10) Pub. No.: US 2022/0056379 A1
(43) Pub. Date: Feb. 24, 2022

(54) Powder Detergent Compositions

(71) Applicant: Novozymes A/S, Bagsvaerd (DK)

(72) Inventors: Astrid Benie, Vaerloese (DK); Carl Mikael Bauer, Malmo (SE)

(73) Assignee: Novozymes A/S, Bagsvaerd (DK)

(21) Appl. No.: 17/299,273

(22) PCT Filed: Dec. 2, 2019
(86) PCT No.: PCT/EP2019/083312

§ 371 Date: Jun. 2, 2021

(30) Foreign Application Priority Data
Dec. 3, 2018 (EP) 18209835.0
Dec. 3, 2018 (EP) 18209836.8

(51) Int. Cl. C11D 3/386; C12N 9/54

(52) U.S. Cl. CPC C11D 3/38609; C12Y 304/21062; C12N 9/54

(57) ABSTRACT

The invention relates to moderate pH and optionally low conductivity powder detergent compositions comprising a protease.

### REFERENCE TO A SEQUENCE LISTING

This application contains a sequence listing in computer readable form, which is incorporated herein by reference.

### FIELD OF THE INVENTION

The present invention relates to moderate pH powder detergent compositions, in particular to moderate pH powder detergent compositions comprising a protease.

### BACKGROUND OF THE INVENTION

Subtilisins are serine proteases from the family S8, in particular from the subfamily S8A, as defined by the MEROPS database (https://www.ebi.ac.uk/merops/index.shtml). In subfamily S8A the key active site residues Asp, His and Ser are typically found in motifs that differ from those of the S8B subfamily.

In the detergent industry, enzymes have for many decades been implemented in washing formulations. Enzymes used in such formulations comprise proteases, lipases, amylases, cellulases, mannosidases as well as other enzymes or mixtures thereof. Commercially, the most important enzymes are proteases.

An increasing number of commercially used proteases for e.g. laundry and dishwashing detergents are protein engineered variants of naturally occurring wild type proteases. Numerous protease variants have been described in the art with alterations relative to a parent protease resulting in improvements such as better wash performance, thermal stability, storage stability or catalytic activity.

Powder detergent formulations are typically highly alkaline with pH values above 9, and often above 10, such as up to about 10.5. Thus, proteases for use in such formulations have generally been designed not only to be able to tolerate high pH values but also to function optimally under alkaline conditions. However, in many cases it is desirable to wash at lower pH values and thus there is a need for proteases that are suitable for and perform well under such conditions.

The present invention provides powder detergent compositions having a lower pH and comprising a protease, wherein the protease shows surprisingly good performance despite the relatively low pH value.

### SUMMARY OF THE INVENTION

The present invention relates to moderate pH powder detergent compositions comprising a protease, where the presence of a protease in the compositions has surprisingly been found to resulting in an improved cleaning performance.

The present invention also relates to use of a composition described herein in a cleaning process, e.g. for laundry or dishwashing, to a method of cleaning using the moderate pH detergent composition, and to use of the proteases described herein in a moderate pH powder detergent composition.

### Overview of Sequences

SEQ ID NO: 1 is the sequence of the Savinase® protease polypeptide from Bacillus lentus.
SEQ ID NO: 2 is the sequence of the BPN′ protease polypeptide from Bacillus amyloliquefaciens.
SEQ ID NO: 3 is the sequence of the TY145 protease polypeptide from Bacillus sp.

### Definitions

Subtilase/protease: The terms “subtilase” and “protease” may be used interchangeably herein and refer to an enzyme that hydrolyses peptide bonds in proteins, i.e. an enzyme with “protease activity”. This includes any enzyme belonging to the EC 3.4 enzyme group (including each of the thirteen subclasses thereof), and in particular endopeptidases (EC 3.4.21). The EC number refers to Enzyme Nomenclature 1992 from NC-IUBMB, Academic Press, San Diego, Calif., including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively.

Protease activity: The term “protease activity” means a proteolytic activity (EC 3.4), in particular endopeptidase activity (EC 3.4.21). There are several protease activity types, the three main activity types being: trypsin-like, where there is cleavage of amide substrates following Arg or Lys at P1, chymotrypsin-like, where cleavage occurs following one of the hydrophobic amino acids at P1, and elastase-like with cleavage following an Ala at P1. Protease activity may be determined according to the procedure described in WO 2016/087619.

Powder detergent composition: The term “powder detergent composition” refers to a detergent composition wherein all or most of the ingredients are in solid dry form. A “powder” typically consists of a mixture comprising one or more powders and/or granulates. The term powder detergent composition includes unit dosage forms such as tabs, which are tablets that have been made by combining, pressing or agglomerating one or more powders or granulates into larger structures in dry form. The water content of a powder detergent composition should be sufficiently low to prevent stickiness and unintended agglomeration of the composition.

The present description and claims will often refer to a “powder” composition for the sake of simplicity. Unless otherwise indicated or apparent from the context, the term “powder” as used herein should be understood to also include solid forms such as granulates and tabs as described above.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Variant: The term “variant” means a polypeptide having protease activity comprising an alteration, i.e., a substitution, insertion, and/or deletion, at one or more positions. A substitution means replacement of the amino acid occupying a position with a different amino acid; a deletion means removal of the amino acid occupying a position; and an insertion means adding an amino acid adjacent to and immediately following the amino acid occupying a position.

### Conventions for Designation of Variants

For purposes of the present invention, the polypeptide of SEQ ID NO: 2 is used to determine the corresponding amino acid residue number in a variant of SEQ ID NO: 1. The amino acid sequence of a variant of SEQ ID NO: 1 is aligned with SEQ ID NO: 2, and based on the alignment, the amino acid position number corresponding to any amino acid residue in the polypeptide of SEQ ID NO: 1. See the paragraph “Numbering of amino acid positions/residues” below for further information.

Numbering of variants of SEQ ID NO: 3 is based on SEQ ID NO: 3.

Identification of the corresponding amino acid residue in another subtilase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

In describing the variants of the present invention, the nomenclature described below is adapted for ease of reference. The accepted IUPAC single letter or three letter amino acid abbreviation is employed. The terms “alteration” or “mutation” may be used interchangeably herein to refer to substitutions, insertions and deletions.

Substitutions. For an amino acid substitution, the following nomenclature is used: Original amino acid, position, substituted amino acid. For example, the substitution of a threonine at position 220 with alanine is designated as “Thr220Ala” or “T220A”. Multiple substitutions may be separated by addition marks (“+”), e.g., “Thr220Ala+Gly229Val” or “T220A+G229V”, representing substitutions at positions 220 and 229 of threonine (T) with alanine (A) and glycine (G) with valine (V), respectively. Multiple substitutions may alternatively be listed with individual mutations separated by a space or a comma. Alternative substitutions in a particular position may be indicated with a slash (“/”). For example, substitution of threonine in position 220 with either alanine, valine or leucine many be designated “T220A/V/L”.

Deletions. For an amino acid deletion, the following nomenclature is used: Original amino acid, position, *. Accordingly, the deletion of threonine at position 220 is designated as “Thr220*” or “T220*”. Multiple deletions may be separated by addition marks (“+”), e.g., “Thr220*+Gly229*” or “T220*+G229*”, or alternatively may be separated by a space or comma. The use of an “X” preceding a position number is as described above for substitutions, e.g. “X131*” means that the amino acid residue at position 131 is deleted.

Insertions. For an amino acid insertion, the following nomenclature is used: Original amino acid, position, original amino acid, inserted amino acid. Accordingly, the insertion of lysine after threonine at position 220 is designated “Thr220ThrLys” or “T220TK”. An insertion of multiple amino acids is designated [Original amino acid, position, original amino acid, inserted amino acid #1, inserted amino acid #2; etc.]. For example, the insertion of lysine and alanine after threonine at position 220 is indicated as “Thr220ThrLysAla” or “T220TKA”.

In such cases the inserted amino acid residue(s) are numbered by the addition of lower-case letters to the position number of the amino acid residue preceding the inserted amino acid residue(s). In the above example, the sequence would thus be:

| Parent: | Variant: |
|---|---|
| 220 | 220 220a 220b |
| T | T - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively. Multiple alterations may alternatively be listed with individual mutations separated by a space or a comma.

A combination of e.g. a substitution and an insertion may be denoted as follows: S99AD, which represents substitution of a serine residue in position 99 with an alanine residue as well as insertion of an aspartic acid residue.

Different alterations. Where different alterations can be introduced at a position, the different alterations may be separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

“Tyr167Gly+Arg170Gly”, “Tyr167Gly+Arg170Ala”, “Tyr167Ala+Arg170Gly”, and “Tyr167Ala+Arg170Ala”.

Different alterations in a position may also be indicated with a slash (“/”), for example “T220A/V/L” as explained above. Alternatively, different alterations may be indicated using brackets, e.g., Arg170[Tyr, Gly] or in one-letter code R170 [Y,G].

Numbering of amino acid positions/residues. The numbering used herein for SEQ ID NO: 1 and SEQ ID NO: 2 is based on the numbering of SEQ ID NO: 2. Thus, for SEQ ID NO: 1, amino acid residues are numbered based on the corresponding amino acid residue in SEQ ID NO: 2. Specifically, the numbering is based on the alignment in Table 1 of WO 89/06279, which shows an alignment of five proteases, including the mature polypeptide of the subtilase BPN′ (BASBPN) sequence (sequence c in the table) and the mature polypeptide of subtilisin 309 from Bacillus lentus, also known as Savinase® (BLSAVI) (sequence a in the table). Persons skilled in the art will know that position numbers used for subtilisin 309 and other proteases in the patent literature are often based on the corresponding position numbers of BPN′ according to this alignment.

For SEQ ID NO: 3 and variants thereof, numbering is based on SEQ ID NO: 3.

### DETAILED DESCRIPTION OF THE INVENTION

The present invention relates in one aspect to a powder detergent composition comprising a protease and at least one detergent component, wherein the composition has a pH of not more than about 9, wherein pH is generally determined in a 5 g/l solution of the composition in deionized water at 20° C.

In a preferred embodiment, the powder detergent composition has a conductivity of not more than about 4.5 mS/cm, preferably not more than about 4.0 mS/cm, wherein conductivity is determined in a 5 g/l solution of the composition in deionized water at 20° C.

Measurement of pH and conductivity in solution is performed using conventional techniques and equipment for pH and conductivity measurements, respectively.

It will be apparent from the present description that the term “moderate pH” as used herein refers to a lower pH relative to conventional powder detergents such as those used for laundry, which as mentioned above typically have a pH in use of above 9 and often above 10.

As indicated above, the term “powder” as used herein is understood to refer to a composition in solid dry form. The “powders” of the invention typically consist of a mixture comprising one or more powders and/or granulates, but also include e.g. unit dosage forms such as tabs.

In one embodiment, the composition of the invention has a pH of below about 9.0, such as not more than about 8.9, such as not more than about 8.8, such as not more than about 8.7, such as not more than about 8.6, such as not more than about 8.5, such as not more than about 8.4, such as not more than about 8.3, such as not more than about 8.2, such as not more than about 8.1, or not more than about 8.0. On the other hand, the composition will generally have a pH of at least about 7, such as at least about 7.1, at least about 7.2, at least about 7.3, at least about 7.4, at least about 7.5, at least about 7.6, at least about 7.7, at least about 7.8, or at least about 7.9. In all cases, pH is determined in a 5 g/l solution as described above.

In some embodiments, the pH may e.g. be in the range of from about 7.0 to not more than about 9.0, for example from about 7.2 to about 8.9, such as from about 7.4 to about 8.8, such as from about 7.6 to about 8.7, such as from about 7.8 to about 8.6.

In one embodiment, the pH may be in the range of from about 7.0 to about 8.2, such as from about 7.2 to about 8.0, determined in a 5 g/l solution as described above.

In another embodiment, the pH may be in the range of from about 7.8 to about 8.8, such as from about 8.0 to about 8.6, determined in a 5 g/l solution as described above.

It should be noted that although, as described above, pH is generally determined in a 5 g/l solution, it is contemplated that for unit dosage forms, e.g. tabs, pH may be determined by dissolving one unit, e.g. one tab, in 15 l of deionized water at 20° C., and measuring the pH of this solution.

In some preferred embodiments, the composition has a conductivity of not more than about 4.0 mS/cm, such as not more than about 3.9 mS/cm, such as not more than about 3.8 mS/cm, such as not more than about 3.7 mS/cm, such as not more than about 3.6 mS/cm, such as not more than about 3.5 mS/cm, such as not more than about 3.4 mS/cm, such as not more than about 3.3 mS/cm, such as not more than about 3.2 mS/cm, such as not more than about 3.1 mS/cm, such as not more than about 3.0 mS/cm, such as not more than about 2.8 mS/cm, such as not more than about 2.6 mS/cm, such as not more than about 2.4 mS/cm, such as not more than about 2.2 mS/cm, or not more than about 2.0 mS/cm.

Similar to the situation for pH, it is contemplated that for unit dosage forms, e.g. tabs, conductivity may be determined by dissolving one unit, e.g. one tab, in 151 of deionized water at 20° C., and measuring the conductivity of this solution.

In one aspect, the invention relates to a moderate pH powder detergent composition wherein the protease is selected from the group consisting of:

a) a variant of the polypeptide of SEQ ID NO: 1, wherein the variant has protease activity and at least 70%, at least 75%, at least 80%, at least 85%, at least 90% or at least 95% but less than 100% sequence identity to SEQ ID NO: 1;

b) the polypeptide of SEQ ID NO: 2 or a variant thereof, wherein the variant has protease activity and at least 70%, at least 75%, at least 80%, at least 85%, at least 90% or at least 95% but less than 100% sequence identity to SEQ ID NO: 2; and

c) the polypeptide of SEQ ID NO: 3 or a variant thereof, wherein the variant has protease activity and at least 70%, at least 75%, at least 80%, at least 85%, at least 90% or at least 95% but less than 100% sequence identity to SEQ ID NO: 3.

In one embodiment, the protease is a variant of the polypeptide of SEQ ID NO: 1 comprising the mutation S99AD, wherein position numbers correspond to positions of the polypeptide of SEQ ID NO: 2, for example a variant having at least 80%, at least 85%, at least 90%, at least 95%, at least 96%, at least 97% or at least 98% sequence identity to SEQ ID NO: 1. In one embodiment, the protease comprises or consists of the polypeptide of SEQ ID NO: 1 with the mutation S99AD.

In one embodiment, the protease is a variant of the polypeptide of SEQ ID NO: 1 comprising the mutations S99D+S103A+V104I+G160S, and optionally one or more additional mutations e.g. selected from S3T, V4I, S101E, S101R, V199M, V105I and L217D, wherein position numbers correspond to positions of the polypeptide of SEQ ID NO: 2, for example a variant having at least 80%, at least 85%, at least 90% or at least 95% sequence identity to SEQ ID NO: 1.

In one such embodiment comprising the mutations S99D+S103A+V104I+G160S, the protease is a variant of the polypeptide of SEQ ID NO: 1 comprising the mutations S3T+V4I+S99D+S101R+S103A+V104I+G160S+V199M+V205I+L217D, for example a variant having at least 80%, at least 85%, at least 90% or at least 95% sequence identity to SEQ ID NO: 1, for example where the protease comprises or consists of the polypeptide of SEQ ID NO: 1 with the mutations S3T+V4I+S99D+S101R+S103A+V104I+G160S+V199M+V205I+L217D.

In another such embodiment comprising the mutations S99D+S103A+V104I+G160S, the protease is a variant of the polypeptide of SEQ ID NO: 1 comprising the mutations S3T+V4I+S99D+S101E+S103A+V104I+G160S+V205I, for example a variant having at least 80%, at least 85%, at least 90% or at least 95% sequence identity to SEQ ID NO: 1, for example where the protease comprises or consists of the polypeptide of SEQ ID NO: 1 with the mutations S3T+V4I+S99D+S101E+S103A+V104I+G160S+V205I.

In another such embodiment comprising the mutations S99D+S103A+V104I+G160S, the protease is a variant of the polypeptide of SEQ ID NO: 1 comprising the mutations S99D+S101E+S103A+V104I+G160S, for example a variant having at least 80%, at least 85%, at least 90%, at least 95% or at least 96% sequence identity to SEQ ID NO: 1, for example where the protease comprises or consists of the polypeptide of SEQ ID NO: 1 with the mutations S99D+S101E+S103A+V104I+G160S.

In another such embodiment comprising the mutations S99D+S103A+V104I+G160S, the protease is a variant of the polypeptide of SEQ ID NO: 1 comprising the mutations S99D+S101E+S103A+V104I+S156D+G160S+L262E, for example a variant having at least 80%, at least 85%, at least 90% or at least 95% sequence identity to SEQ ID NO: 1, for example where the protease comprises or consists of the polypeptide of SEQ ID NO: 1 with the mutations S99D+S101E+S103A+V104I+S156D+G160S+L262E.

In one embodiment, the protease is a variant of the polypeptide of SEQ ID NO: 1 comprising the mutation S99SE, wherein position numbers correspond to positions of the polypeptide of SEQ ID NO: 2, for example a variant having at least 80%, at least 85%, at least 90%, at least 95%, at least 96%, at least 97% or at least 98% sequence identity to SEQ ID NO: 1. In one embodiment, the protease comprises or consists of the polypeptide of SEQ ID NO: 1 with the mutation S99SE.

In one embodiment, the protease is a variant of the polypeptide of SEQ ID NO: 1 comprising the mutations Y167A+R170S+A194P, wherein position numbers correspond to positions of the polypeptide of SEQ ID NO: 2, for example a variant having at least 80%, at least 85%, at least 90%, at least 95%, at least 96%, at least 97% or at least 98% sequence identity to SEQ ID NO: 1. In one embodiment, the protease comprises or consists of the polypeptide of SEQ ID NO: 1 with the mutations Y167A+R170S+A194P.

In one embodiment, the protease is a variant of the polypeptide of SEQ ID NO: 1 comprising three or more mutations selected from the group consisting of S9E, N43R, N76D, V205I, Q206L, Y209W, S259D, N261W and L262E, for example 4, 5, 6, 7 or 8 of said mutations, wherein position numbers correspond to positions of the polypeptide of SEQ ID NO: 2, for example a variant having at least 80%, at least 85%, at least 90% or at least 95% sequence identity to SEQ ID NO: 1. In a particular embodiment, the protease may be a variant of the polypeptide of SEQ ID NO: 1 comprising the mutations S9E+N43R+N76D+V205I+Q206L+Y209W+S259D+N261W+L262E, wherein position numbers correspond to positions of the polypeptide of SEQ ID NO: 2, for example a variant having at least 80%, at least 85%, at least 90% or at least 95% sequence identity to SEQ ID NO: 1. In one embodiment, the protease comprises or consists of the polypeptide of SEQ ID NO: 1 with the mutations S9E+N43R+N76D+V205I+Q206L+Y209W+S259D+N261W+L262E.

In one embodiment, the protease is a variant of the polypeptide of SEQ ID NO: 1 comprising the mutations S87N+S101G+V104N, wherein position numbers correspond to positions of the polypeptide of SEQ ID NO: 2, for example a variant having at least 80%, at least 85%, at least 90%, at least 95%, at least 96%, at least 97% or at least 98% sequence identity to SEQ ID NO: 1. In one embodiment, the protease comprises or consists of the polypeptide of SEQ ID NO: 1 with the mutations S87N+S101G+V104N.

In one embodiment, the protease comprises or consists of the polypeptide of SEQ ID NO: 2.

In one embodiment, the protease is a variant of the polypeptide of SEQ ID NO: 2 comprising the mutation Y217L, for example a variant having at least 80%, at least 85%, at least 90%, at least 95%, at least 96%, at least 97% or at least 98% sequence identity to SEQ ID NO: 2. In one embodiment, the protease comprises or consists of the polypeptide of SEQ ID NO: 2 with the mutation Y217L.

In one embodiment, the protease is a variant of the polypeptide of SEQ ID NO: 2 comprising the mutations S24G+S53G+S78N+S101N+G128S+Y217Q, for example a variant having at least 80%, at least 85%, at least 90%, at least 95% or at least 96% sequence identity to SEQ ID NO: 2. In one embodiment, the protease comprises or consists of the polypeptide of SEQ ID NO: 2 with the mutations S24G+S53G+S78N+S101N+G128S+Y217Q.

In one embodiment, the protease is a variant of the polypeptide of SEQ ID NO: 2 comprising the mutations S24G+S53G+S78N+S101N+G128A+Y217Q, for example a variant having at least 80%, at least 85%, at least 90%, at least 95% or at least 96% sequence identity to SEQ ID NO: 2. In one embodiment, the protease comprises or consists of the polypeptide of SEQ ID NO: 2 with the mutations S24G+S53G+S78N+S101N+G128A+Y217Q.

In one embodiment, the protease comprises or consists of the polypeptide of SEQ ID NO: 3.

In one embodiment, the protease is a variant of the polypeptide of SEQ ID NO: 3 having at least 80%, at least 85%, at least 90% or at least 95% sequence identity to SEQ ID NO: 3. The protease may e.g. be a variant of the polypeptide of SEQ ID NO: 3 comprising one or more mutations selected from the group consisting of S27K, N109K, S111E, S171E, S173P, G174K, S175P, F180Y, G182A, L184F, Q198E, N199K and T297P, for example 1, 2, 3, 4, 5, 6, 7, 8, 9, 10, 11, 12 or all of said mutations.

In one embodiment, the protease is a variant of the polypeptide of SEQ ID NO: 3 comprising the mutations S27K+N109K+S111E+S171E+S173P+G174K+S175P+F180Y+G182A+L184F+Q198E+N199K+T297P, for example a variant having at least 80%, at least 85%, at least 90% or at least 95% sequence identity to SEQ ID NO: 3. In one embodiment, the protease comprises or consists of the polypeptide of SEQ ID NO: 3 with the mutations S27K+N109K+S111E+S171E+S173P+G174K+S175P+F180Y+G182A+L184F+Q198E+N199K+T297P.

In some embodiments, it is believed to be sufficient that the composition has a pH as specified above, i.e. a low conductivity is not necessarily required. Non-limiting examples of such embodiments are provided below.

In one embodiment, the composition has a pH, determined as described above, of from about 7.0 to not more than about 9.0, for example from about 7.2 to about 8.9, such as from about 7.4 to about 8.8, and the protease is a variant of the polypeptide of SEQ ID NO: 1 comprising the mutation S99AD, wherein position numbers correspond to positions of the polypeptide of SEQ ID NO: 2, for example a variant having at least 80%, at least 85%, at least 90%, at least 95%, at least 96%, at least 97% or at least 98% sequence identity to SEQ ID NO: 1; for example where the protease comprises or consists of the polypeptide of SEQ ID NO: 1 with the mutation S99AD.

In one embodiment, the composition has a pH, determined as described above, of from about 7.0 to not more than about 9.0, for example from about 7.2 to about 8.9, such as from about 7.4 to about 8.8, and the protease is a variant of the polypeptide of SEQ ID NO: 1 comprising the mutations S99D+S103A+V104I+G160S, and optionally one or more additional mutations e.g. selected from S3T, V4I, S101E, S101R, V199M, V105I and L217D, wherein position numbers correspond to positions of the polypeptide of SEQ ID NO: 2, for example a variant having at least 80%, at least 85%, at least 90% or at least 95% sequence identity to SEQ ID NO: 1.

In one such embodiment comprising the mutations S99D+S103A+V104I+G160S, the composition has a pH, determined as described above, of from about 7.0 to not more than about 9.0, for example from about 7.2 to about 8.9, such as from about 7.4 to about 8.8, and the protease is a variant of the polypeptide of SEQ ID NO: 1 comprising the mutations S3T+V4I+S99D+S101R+S103A+V104I+G160S+V199M+V205I+L217D, for example a variant having at least 80%, at least 85%, at least 90% or at least 95% sequence identity to SEQ ID NO: 1; for example where the protease comprises or consists of the polypeptide of SEQ ID NO: 1 with the mutations S3T+V4I+S99D+S101R+S103A+V104I+G160S+V199M+V205I+L217D.

In another such embodiment comprising the mutations S99D+S103A+V104I+G160S, the composition has a pH, determined as described above, of from about 7.0 to not more than about 9.0, for example from about 7.2 to about 8.9, such as from about 7.4 to about 8.8, and the protease is a variant of the polypeptide of SEQ ID NO: 1 comprising the mutations S3T+V4I+S99D+S101E+S103A+V104I+G160S+V205I, for example a variant having at least 80%, at least 85%, at least 90% or at least 95% sequence identity to SEQ ID NO: 1; for example where the protease comprises or consists of the polypeptide of SEQ ID NO: 1 with the mutations S3T+V4I+S99D+S101E+S103A+V104I+G160S+V205I.

In another such embodiment comprising the mutations S99D+S103A+V104I+G160S, the composition has a pH, determined as described above, of from about 7.0 to not more than about 9.0, for example from about 7.2 to about 8.9, such as from about 7.4 to about 8.8, and the protease is a variant of the polypeptide of SEQ ID NO: 1 comprising the mutations S99D+S101E+S103A+V104I+G160S, for example a variant having at least 80%, at least 85%, at least 90%, at least 95% or at least 96% sequence identity to SEQ ID NO: 1; for example where the protease comprises or consists of the polypeptide of SEQ ID NO: 1 with the mutations S99D+S101E+S103A+V104I+G160S.

In one embodiment, the composition has a pH, determined as described above, of from about 7.0 to not more than about 9.0, for example from about 7.2 to about 8.9, such as from about 7.4 to about 8.8, and the protease is a variant of the polypeptide of SEQ ID NO: 1 comprising the mutation S99SE, wherein position numbers correspond to positions of the polypeptide of SEQ ID NO: 2, for example a variant having at least 80%, at least 85%, at least 90%, at least 95%, at least 96%, at least 97% or at least 98% sequence identity to SEQ ID NO: 1; for example where the protease comprises or consists of the polypeptide of SEQ ID NO: 1 with the mutation S99SE.

In one embodiment, the composition has a pH, determined as described above, of from about 7.0 to not more than about 9.0, for example from about 7.2 to about 8.9, such as from about 7.4 to about 8.8, and the protease is a variant of the polypeptide of SEQ ID NO: 1 comprising the mutations Y167A+R170S+A194P, wherein position numbers correspond to positions of the polypeptide of SEQ ID NO: 2, for example a variant having at least 80%, at least 85%, at least 90%, at least 95%, at least 96%, at least 97% or at least 98% sequence identity to SEQ ID NO: 1; for example where the protease comprises or consists of the polypeptide of SEQ ID NO: 1 with the mutations Y167A+R170S+A194P.

In one embodiment, the composition has a pH, determined as described above, of from about 7.0 to not more than about 9.0, for example from about 7.2 to about 8.9, such as from about 7.4 to about 8.8, and the protease is a variant of the polypeptide of SEQ ID NO: 1 comprising three or more mutations selected from the group consisting of S9E, N43R, N76D, V205I, Q206L, Y209W, S259D, N261W and L262E, for example 4, 5, 6, 7 or 8 of said mutations, wherein position numbers correspond to positions of the polypeptide of SEQ ID NO: 2, for example a variant having at least 80%, at least 85%, at least 90% or at least 95% sequence identity to SEQ ID NO: 1. In a particular embodiment, the protease may be a variant of the polypeptide of SEQ ID NO: 1 comprising the mutations S9E+N43R+N76D+V205I+Q206L+Y209W+S259D+N261W+L262E, wherein position numbers correspond to positions of the polypeptide of SEQ ID NO: 2, for example a variant having at least 80%, at least 85%, at least 90% or at least 95% sequence identity to SEQ ID NO: 1; for example where the protease comprises or consists of the polypeptide of SEQ ID NO: 1 with the mutations S9E+N43R+N76D+V205I+Q206L+Y209W+S259D+N261W+L262E.

In one embodiment, the composition has a pH, determined as described above, of from about 7.0 to not more than about 9.0, for example from about 7.2 to about 8.9, such as from about 7.4 to about 8.8, and the protease is a variant of the polypeptide of SEQ ID NO: 1 comprising the mutations S87N+S101G+V104N, wherein position numbers correspond to positions of the polypeptide of SEQ ID NO: 2, for example a variant having at least 80%, at least 85%, at least 90%, at least 95%, at least 96%, at least 97% or at least 98% sequence identity to SEQ ID NO: 1; for example where the protease comprises or consists of the polypeptide of SEQ ID NO: 1 with the mutations S87N+S101G+V104N.

In any of the embodiments above wherein the composition has a pH of not more than about 9, the composition preferably has an improved wash performance compared to a reference composition having a pH of 10, wherein pH is determined in a 5 g/l solution in deionized water at 20° C. Wash performance may e.g. be determined using the AMSA assay as described in the examples below.

In other embodiments, it is preferred that the composition has both a pH as specified above as well as a low conductivity. Non-limiting examples of such embodiments are provided below.

In one embodiment, the composition has a pH, determined as described above, of from about 7.0 to not more than about 9.0, for example from about 7.2 to about 8.9, such as from about 7.4 to about 8.8, and a conductivity, determined as described above, of not more than about 4.0 mS/cm, such as not more than about 3.8 mS/cm, such as not more than about 3.6 mS/cm, such as not more than about 3.4 mS/cm, such as not more than about 3.2 mS/cm, such as not more than about 3.0 mS/cm, such as not more than about 2.5 mS/cm, or not more than about 2.0 mS/cm, and the protease is a variant of the polypeptide of SEQ ID NO: 1 comprising the mutations S99D+S101E+S103A+V104I+S156D+G160S+L262E, wherein position numbers correspond to positions of the polypeptide of SEQ ID NO: 2, for example a variant having at least 80%, at least 85%, at least 90% or at least 95% sequence identity to SEQ ID NO: 1; for example where the protease comprises or consists of the polypeptide of SEQ ID NO: 1 with the mutations S99D+S101E+S103A+V104I+S156D+G160S+L262E.

In one embodiment, the composition has a pH, determined as described above, of from about 7.0 to not more than about 9.0, for example from about 7.2 to about 8.9, such as from about 7.4 to about 8.8, and a conductivity, determined as described above, of not more than about 4.0 mS/cm, such as not more than about 3.8 mS/cm, such as not more than about 3.6 mS/cm, such as not more than about 3.4 mS/cm, such as not more than about 3.2 mS/cm, such as not more than about 3.0 mS/cm, such as not more than about 2.5 mS/cm, or not more than about 2.0 mS/cm, and the protease comprises or consists of the polypeptide of SEQ ID NO: 2.

In one embodiment, the composition has a pH, determined as described above, of from about 7.0 to not more than about 9.0, for example from about 7.2 to about 8.9, such as from about 7.4 to about 8.8, and a conductivity, determined as described above, of not more than about 4.0 mS/cm, such as not more than about 3.8 mS/cm, such as not more than about 3.6 mS/cm, such as not more than about 3.4 mS/cm, such as not more than about 3.2 mS/cm, such as not more than about 3.0 mS/cm, such as not more than about 2.5 mS/cm, or not more than about 2.0 mS/cm, and the protease is a variant of the polypeptide of SEQ ID NO: 2 comprising the mutation Y217L, for example a variant having at least 80%, at least 85%, at least 90%, at least 95%, at least 96%, at least 97% or at least 98% sequence identity to SEQ ID NO: 2; for example where the protease comprises or consists of the polypeptide of SEQ ID NO: 2 with the mutation Y217L.

In one embodiment, the composition has a pH, determined as described above, of from about 7.0 to not more than about 9.0, for example from about 7.2 to about 8.9, such as from about 7.4 to about 8.8, and a conductivity, determined as described above, of not more than about 4.0 mS/cm, such as not more than about 3.8 mS/cm, such as not more than about 3.6 mS/cm, such as not more than about 3.4 mS/cm, such as not more than about 3.2 mS/cm, such as not more than about 3.0 mS/cm, such as not more than about 2.5 mS/cm, or not more than about 2.0 mS/cm, and the protease is a variant of the polypeptide of SEQ ID NO: 2 comprising the mutations S24G+S53G+S78N+S101N+G128S+Y217Q, for example a variant having at least 80%, at least 85%, at least 90%, at least 95% or at least 96% sequence identity to SEQ ID NO: 2; for example where the protease comprises or consists of the polypeptide of SEQ ID NO: 2 with the mutations S24G+S53G+S78N+S101N+G128S+Y217Q.

In one embodiment, the composition has a pH, determined as described above, of from about 7.0 to not more than about 9.0, for example from about 7.2 to about 8.9, such as from about 7.4 to about 8.8, and a conductivity, determined as described above, of not more than about 4.0 mS/cm, such as not more than about 3.8 mS/cm, such as not more than about 3.6 mS/cm, such as not more than about 3.4 mS/cm, such as not more than about 3.2 mS/cm, such as not more than about 3.0 mS/cm, such as not more than about 2.5 mS/cm, or not more than about 2.0 mS/cm, and the protease is a variant of the polypeptide of SEQ ID NO: 2 comprising the mutations S24G+S53G+S78N+S101N+G128A+Y217Q, for example a variant having at least 80%, at least 85%, at least 90%, at least 95% or at least 96% sequence identity to SEQ ID NO: 2; for example where the protease comprises or consists of the polypeptide of SEQ ID NO: 2 with the mutations S24G+S53G+S78N+S101N+G128A+Y217Q.

In one embodiment, the composition has a pH, determined as described above, of from about 7.0 to not more than about 9.0, for example from about 7.2 to about 8.9, such as from about 7.4 to about 8.8, and a conductivity, determined as described above, of not more than about 4.0 mS/cm, such as not more than about 3.8 mS/cm, such as not more than about 3.6 mS/cm, such as not more than about 3.4 mS/cm, such as not more than about 3.2 mS/cm, such as not more than about 3.0 mS/cm, such as not more than about 2.5 mS/cm, or not more than about 2.0 mS/cm, and the protease is a variant of the polypeptide of SEQ ID NO: 3 having at least 80%, at least 85%, at least 90% or at least 95% sequence identity to SEQ ID NO: 3; for example where the protease is a variant of the polypeptide of SEQ ID NO: 3 comprising one or more mutations selected from the group consisting of S27K, N109K, S111E, S171E, S173P, G174K, S175P, F180Y, G182A, L184F, Q198E, N199K and T297P, for example 1, 2, 3, 4, 5, 6, 7, 8, 9, 10, 11, 12 or all of said mutations.

In one embodiment, the composition has a pH, determined as described above, of from about 7.0 to not more than about 9.0, for example from about 7.2 to about 8.9, such as from about 7.4 to about 8.8, and a conductivity, determined as described above, of not more than about 4.0 mS/cm, such as not more than about 3.8 mS/cm, such as not more than about 3.6 mS/cm, such as not more than about 3.4 mS/cm, such as not more than about 3.2 mS/cm, such as not more than about 3.0 mS/cm, such as not more than about 2.5 mS/cm, or not more than about 2.0 mS/cm, and the protease is a variant of the polypeptide of SEQ ID NO: 3 comprising the mutations S27K+N109K+S111E+S171E+S173P+G174K+S175P+F180Y+G182A+L184F+Q198E+N199K+T297P, for example a variant having at least 80%, at least 85%, at least 90% or at least 95% sequence identity to SEQ ID NO: 3; for example where the protease comprises or consists of the polypeptide of SEQ ID NO: 3 with the mutations S27K+N109K+S111E+S171E+S173P+G174K+S175P+F180Y+G182A+L184F+Q198E+N199K+T297P.

In any of the embodiments herein wherein the composition has a pH of not more than about 9 and a conductivity of not more than about 4.0 mS/cm, the composition preferably has an improved wash performance compared to a reference composition having a conductivity of 4.2 mS/cm, and preferably compared to a reference composition having a conductivity of 4.5 mS/cm, wherein pH and conductivity are determined in a 5 g/l solution in deionized water at 20° C. Wash performance may e.g. be determined using the AMSA assay as described in the examples below.

In addition to the amino acid alterations specifically disclosed herein, a protease variant in a composition of the invention may comprise additional alterations at one or more other positions. These additional alterations may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, in The Proteins, Academic Press, New York. Common conservative substitution groups include, but are not limited to: G=A=S; I=V=L=M; D=E; Y=F; and N=Q (where e.g. “G=A=S” means that these three amino acids may be substituted for each other).

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for protease activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

### Detergent Compositions

In one embodiment, the invention relates to a moderate pH and preferably low conductivity powder composition as described above comprising a protease and further comprising one or more additional enzymes selected from the group consisting of amylases, catalases, cellulases (e.g., endoglucanases), cutinases, haloperoxygenases, lipases, mannanases, pectinases, pectin lyases, peroxidases, proteases, xanthanases, lichenases and xyloglucanases, or any mixture thereof.

The detergent composition may e.g. be in the form of a regular or compact powder, a granulate, a homogeneous tablet, or a tablet having two or more layers. The composition, e.g. powder, granulate or tablet, may also form part of a composite composition such as a compartment in a multiple compartment pouch or pod.

The invention also relates to use of a composition of the present in a cleaning process, such as laundry or hard surface cleaning such as dish wash.

The choice of additional components for a detergent composition is within the skill of the artisan and includes conventional ingredients, including the exemplary non-limiting components set forth below. The choice of components may include, for fabric care, the consideration of the type of fabric to be cleaned, the type and/or degree of soiling, the temperature at which cleaning is to take place, and the formulation of the detergent product.

In a particular embodiment, a detergent composition comprises a protease and one or more non-naturally occurring detergent components, such as surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers. The detergent composition will typically comprise at least a surfactant and a builder.

In one embodiment, the protease may be added to a detergent composition in an amount corresponding to 0.01-200 mg of enzyme protein per liter of wash liquor, preferably 0.05-50 mg of enzyme protein per liter of wash liquor, in particular 0.1-10 mg of enzyme protein per liter of wash liquor.

A granulated composition for laundry may for example include 0.001%-20%, such as 0.01%-10%, such as 0.05%-5% of enzyme protein by weight of the composition.

An automatic dish wash (ADW) composition may for example include 0.001%-30%, such as 0.01%-20%, such as 0.1-15%, such as 0.5-10% of enzyme protein by weight of the composition.

The enzymes such as the protease may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in, for example, WO 92/19709 and WO 92/19708 or the protease may be stabilized using peptide aldehydes or ketones such as described in WO 2005/105826 and WO 2009/118375.

The detergent composition may be formulated into a granular detergent for laundry. Such detergent may e.g. comprise;
a) at least 0.01 mg protease per gram of composition
b) anionic surfactant, preferably 5 wt % to 50 wt %
c) nonionic surfactant, preferably 1 wt % to 8 wt %
d) builder, preferably 5 wt % to 40 wt %, such as carbonates, zeolites, phosphate builder, calcium sequestering builders or complexing agents.

Although components mentioned below are categorized by general header according to a particular functionality, this is not to be construed as a limitation, as a component may comprise additional functionalities as will be appreciated by the person skilled in the art.

Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized. Surfactants lower the surface tension in the detergent, which allows the stain being cleaned to be lifted and dispersed and then washed away.

When included therein, the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein, the detergent will usually contain from about 0% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein, the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein, the detergent will usually contain from about 0% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein, the detergent will usually contain from about 0% to about 10% by weight of a zwitterionic surfactant. Non-limiting examples of zwitterionic surfactants include betaine, alkyldimethylbetaine, sulfobetaine, and combinations thereof.

Builders and Co-Builders

The detergent composition may contain about 0-65% by weight, such as about 5% to about 45% of a detergent builder or co-builder, or a mixture thereof. In a dish wash detergent, the level of builder is typically 40-65%, particularly 50-65%. Builders and chelators soften, e.g., the wash water by removing the metal ions form the liquid. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

In a preferred embodiment, the detergent composition is phosphate-free.

The detergent composition may also contain 0-20% by weight, such as about 5% to about 10%, of a detergent co-builder, or a mixture thereof. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra-(methylenephosphonic acid) (EDTMPA), diethylenetriaminepentakis (methylenephosphonic acid) (DTPMPA or DTMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 2009/102854 and U.S. Pat. No. 5,977,053.

The subtilase variants of the invention may also be formulated into a dish wash composition, preferably an automatic dish wash composition (ADW), comprising:

a) at least 0.01 mg of active protease variant according to the invention, and

b) 10-50 wt % builder preferably selected from citric acid, methylglycine-N,N-diacetic acid (MGDA) and/or glutamic acid-N,N-diacetic acid (GLDA) and mixtures thereof, and

c) at least one bleach component.

### Bleaching Systems

The detergent may contain 0-50% by weight, such as about 0.1% to about 25%, of a bleaching system. Bleach systems remove discolor often by oxidation, and many bleaches also have strong bactericidal properties, and are used for disinfecting and sterilizing. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone®, and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator.

The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO 98/17767. A particular family of bleach activators of interest was disclosed in EP 624154 and particularly preferred in that family is acetyl triethyl citrate (ATC). ATC or a short chain triglyceride like triacetin has the advantage that it is environmentally friendly as it eventually degrades into citric acid and alcohol. Furthermore, acetyl triethyl citrate and triacetin have good hydrolytic stability in the product upon storage and are efficient bleach activators. Finally, ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst or a booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-KN-methanylylidene)triphenolato-κ3O]manganese(III). The bleach catalysts may also be other metal compounds, such as iron or cobalt complexes.

In some embodiments, the bleach component may be an organic catalyst selected from the group consisting of organic catalysts having the following formula:

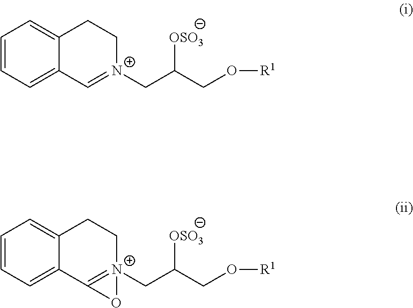

(iii) and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g., in WO 2007/087258, WO 2007/087244, WO 2007/087259 and WO 2007/087242. Suitable photobleaches may for example be sulfonated zinc phthalocyanine.

Hydrotropes

A hydrotrope is a compound that solubilizes hydrophobic compounds in aqueous solutions (or oppositely, polar substances in a non-polar environment). Typically, hydrotropes have both hydrophilic and hydrophobic characters (so-called amphiphilic properties as known from surfactants); however, the molecular structures of hydrotropes generally do not favour spontaneous self-aggregation, see, e.g., review by Hodgdon and Kaler, 2007, Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behaviour, stability, and colloidal properties of systems containing substances of polar and non-polar character, including mixtures of water, oil, surfactants, and polymers. Hydrotropes are classically used across industries from pharma, personal care and food to technical applications. Use of hydrotropes in detergent compositions allows for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

Fabric Hueing Agents

The detergent compositions of the present invention may also include fabric hueing agents such as dyes or pigments, which when formulated in detergent compositions can deposit onto a fabric when the fabric is contacted with a wash liquor comprising the detergent compositions and thus altering the tint of the fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/003274, WO 2005/003275, WO 2005/003276 and EP 1876226 (hereby incorporated by reference). The detergent composition preferably comprises from about 0.00003 wt. % to about 0.2 wt. %, from about 0.00008 wt. % to about 0.05 wt. %, or even from about 0.0001 wt. % to about 0.04 wt. % fabric hueing agent. The composition may comprise from 0.0001 wt % to 0.2 wt. % fabric hueing agent, this may be especially preferred when the composition is in the form of a unit dose pouch. Suitable hueing agents are also disclosed in, e.g., WO 2007/087257 and WO 2007/087243.

Additional Enzymes

A detergent additive or detergent composition may comprise one or more enzymes such as an amylase, arabinase, carbohydrase, cellulase (e.g., endoglucanase), cutinase, galactanase, haloperoxygenase, lipase, mannanase, oxidase, e.g., laccase and/or peroxidase, pectinase, pectin lyase, protease, xylanase, xanthanase or xyloglucanase.

The properties of the selected enzyme(s) should be compatible with the selected detergent (e.g. pH-optimum, compatibility with other enzymatic and non-enzymatic ingredients, etc.).

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having color care benefits. Examples of such cellulases are cellulases described in EP 495257, EP 531372, WO 96/11262, WO 96/29397, WO 98/08940. Other examples are cellulase variants such as those described in WO 94/07998, EP 531315, U.S. Pat. Nos. 5,457,046, 5,686,593, 5,763,254, WO 95/24471, WO 98/12307 and PCT/DK98/00299.

Examples of cellulases exhibiting endo-beta-1,4-glucanase activity (EC 3.2.1.4) are described in WO 02/99091.

Other examples of cellulases include the family 45 cellulases described in WO 96/29397, and especially variants thereof having a substitution, insertion and/or deletion at one or more of the positions corresponding to the following positions in SEQ ID NO: 8 of WO 02/99091:2, 4, 7, 8, 10, 13, 15, 19, 20, 21, 25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113, 114, 117, 119, 121, 133, 136, 137, 138, 139, 140a, 141, 143a, 145, 146, 147, 150e, 150j, 151, 152, 153, 154, 155, 156, 157, 158, 159, 160c, 160e, 160k, 161, 162, 164, 165, 168, 170, 171, 172, 173, 175, 176, 178, 181, 183, 184, 185, 186, 188, 191, 192, 195, 196, 200, and/or 20, preferably selected among P19A, G20K, Q44K, N48E, Q119H or Q146R.

Commercially available cellulases include Celluzyme®, Carezyme® and Celluclean® (Novozymes A/S), Clazinase™, and Puradax HA™ (Genencor International Inc.), and KAC-500(B)™ (Kao Corporation).

Proteases

The composition may comprise one or more additional proteases including those of bacterial, fungal, plant, viral or animal origin, e.g., vegetable or microbial origin. Microbial origin is preferred. Chemically modified or protein engineered mutants are included. It may be an alkaline protease, such as a serine protease or a metalloprotease. A serine protease may for example be of the 51 family, such as trypsin, or the S8 family such as subtilisin. A metalloprotease may for example be a thermolysin from, e.g., family M4 or other metalloprotease such as those from M5, M7 or M8 families.

Examples of metalloproteases are the neutral metalloproteases as described in WO 2007/044993 (Genencor Int.) such as those derived from Bacillus amyloliquefaciens.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase®, Esperase®, Progress® Uno, Progress® In and Progress® Excel (Novozymes A/S), those sold under the tradename Maxatase®, Maxacal®, Maxapem®, Purafect®, Purafect Prime®, Purafect MA®, Purafect Ox®, Purafect OxP®, Puramax®, Properase®, FN2®, FN3®, FN4®, Excellase®, Excellenz™ P1000, Excellenz™ P1250, Eraser®, Preferenz® P100, Preferenz® P300, Purafect Prime, Preferenz P110™, Effectenz P1000™, Purafect®, Effectenz P1050™, Purafect® Ox, Effectenz™ P2000, Purafast™, Properase®, Opticlean™ and Optimase® Opticlean® and Optimase® (Danisco/DuPont), Axapem™ (Gist-Brocades N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases

Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Humicola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 2007/87508 and WO 2009/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g., acyltransferases with homology to Candida antarctica lipase A (WO 2010/111143), acyltransferase from Mycobacterium smegmatis (WO 2005/056782), perhydrolases from the CE 7 family (WO 2009/067279), and variants of the M. smegmatis perhydrolase, in particular the S54V variant used in the commercial product Gentle Power Bleach from Huntsman Textile Effects Pte Ltd (WO 2010/100028).

Amylases

Suitable amylases which can be used together with the protease may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/19467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/10355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylases comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T491+G107A+H156Y+A181T+N190F+1201F+A209V+Q264S.

Other suitable amylases are amylases having the sequence of SEQ ID NO: 6 in WO 99/19467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/23873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/23873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 2008/153815, SEQ ID NO: 10 in WO 01/66712 or variants thereof having 90% sequence identity to SEQ ID NO: 2 of WO 2008/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 2009/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K,

wherein the variants are C-terminally truncated and optionally further comprise a substitution at position 243 and/or a deletion at position 180 and/or position 181.

Further suitable amylases are amylases having SEQ ID NO: 1 of WO 2013/184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or a deletion in position R178 and/or S179 or of T180 and/or G181. Most preferred amylase variants of SEQ ID NO: 1 comprise the substitutions:

E187P+I203Y+G476K

E187P+I203Y+R458N+T459S+D460T+G476K

and optionally further comprise a substitution at position 241 and/or a deletion at position 178 and/or position 179.

Further suitable amylases are amylases having SEQ ID NO: 1 of WO 2010/104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478.

More preferred variants of SEQ ID NO: 1 are those having the substitution in one of more of the following positions: N21D, D97N, V128I K177L, M200L, L204YF, E242QA, G477K and G478K and/or a deletion in position R179 and/or S180 or of 1181 and/or G182. Most preferred amylase variants of SEQ ID NO: 1 comprise the substitutions N21D+D97N+V128I, and optionally further comprise a substitution at position 200 and/or a deletion at position 180 and/or position 181.

Other suitable amylases are the alpha-amylase having SEQ ID NO: 12 in WO 01/66712 or a variant having at least 90% sequence identity to SEQ ID NO: 12. Preferred amylase variants are those having a substitution, a deletion or an insertion in one of more of the following positions of SEQ ID NO: 12 in WO 01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particularly preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087. Commercially available amylases include Duramyl™, Termamyl™, Fungamyl™, Stainzyme™, Stainzyme Plus™, Natalase™, Liquozyme X, BAN™, Amplify® and Amplify® Prime (from Novozymes A/S), and Rapidase™, Purastar™/Effectenz™, Powerase, Preferenz S1000, Preferenz S100 and Preferenz S110 (from Genencor International Inc./DuPont).

One preferred amylase is a variant of the amylase having SEQ ID NO: 13 in WO 2016/180748 with the alterations H1*+N54S+V56T+K72R+G109A+F113Q+R116Q+W167F+Q172G+A174S+G182*+D183*+G184T+N195F+V206L+K391A+P473R+G476K.

Another preferred amylase is a variant of the amylase having SEQ ID NO: 1 in WO 2013/001078 with the alterations D183*+G184*+W140Y+N195F+V206Y+Y243F+E260G+G304R+G476K.

Another preferred amylase is a variant of the amylase having SEQ ID NO: 1 in WO 2018/141707 with the alterations H1*+G7A+G109A+W140Y+G182*+D183*+N195F+V206Y+Y243F+E260G+N280S+G304R+E391A+G476K.

A further preferred amylase is a variant of the amylase having SEQ ID NO: 1 in WO 2017/191160 with the alterations L202M+T246V.

Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes A/S).

Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants: The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant Science Series, volume 71, Marcel Dekker, Inc., 1997.

Dye Transfer Inhibiting Agents: The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent: The detergent compositions of the present invention will preferably also contain additional components that may tint articles being cleaned, such as fluorescent whitening agent or optical brighteners. Where present the brightener is preferably at a level of about 0.01% to about 05%. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2.2′-disulphonate; 4,4′-bis-(2-anilino-4(N-methyl-N-2-hydroxy-ethylamino)-s-triazin-6-ylamino) stilbene-2,2′-disulphonate, 4,4′-bis-(4-phenyl-2,1,3-triazol-2-yl)stilbene-2,2′-disulphonate; 4,4′-bis-(2-anilino-4(1-methyl-2-hydroxy-ethylamino)-s-triazin-6-ylamino) stilbene-2,2′-disulphonate and 2-(stilbyl-4″-naptho-1,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt. % to upper levels of 0.5 or even 0.75 wt. %.

Soil release polymers: The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalate based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 03/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents: The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

Formulation of Detergent Products

The detergent enzyme(s), i.e. a protease and optionally one or more additional enzymes, may be included in a detergent composition by adding separate additives containing one or more enzymes, or by adding a combined additive comprising these enzymes. A detergent additive comprising one or more enzymes can be formulated, for example, as a granulate, in particular a non-dusting granulate.

The detergent composition of the invention may be in any convenient form, e.g., a regular or compact powder, a granulate, a homogenous tablet, a tablet having two or more layers. The powder composition, e.g. powder, granulate or tablet, may also form part of a composite composition such as a compartment in a multiple compartment pouch or pod.

Pouches (pods) can be configured as single or multiple compartments and can be of any form, shape and material suitable to hold the composition, without allowing the release of the composition from the pouch prior to water contact. The pouch is made from water soluble film which encloses an inner volume. The inner volume can be divided into compartments of the pouch. Preferred films are polymeric materials, preferably polymers which are formed into a film or sheet. Preferred polymers, copolymers or derivates thereof are selected from polyacrylates, and water-soluble acrylate copolymers, methyl cellulose, carboxy methyl cellulose, sodium dextrin, ethyl cellulose, hydroxyethyl cellulose, hydroxypropyl methyl cellulose, maltodextrin, polymethacrylates, most preferably polyvinyl alcohol copolymers and hydroxypropyl methyl cellulose (HPMC). Preferably the level of polymer in the film for example PVA is at least about 60%. The preferred average molecular weight will typically be about 20,000 to about 150,000. Films can also be of blend compositions comprising hydrolytically degradable and water-soluble polymer blends such as polylactide and polyvinyl alcohol (known under the Trade reference M8630 as sold by Chris Craft In. Prod. of Gary, Ind., US) plus plasticizers like glycerol, ethylene glycerol, propylene glycol, sorbitol and mixtures thereof. The pouches can for example comprise a solid laundry detergent composition or part components and/or a liquid cleaning composition or part components separated by the water-soluble film. The compartment for liquid components can be different in composition than compartments containing solids. See, e.g., US 2009/0011970.

Detergent ingredients can be separated physically from each other by compartments in water dissolvable pouches or in different layers of tablet, thereby avoiding negative storage interaction between components. Different dissolution profiles of each of the compartments can also give rise to delayed dissolution of selected components in the wash solution.

Granular Detergent Formulations

Enzymes in the form of granules, comprising an enzyme-containing core and optionally one or more coatings, are commonly used in granular (powder) detergents. Various methods for preparing the core are well-known in the art and include, for example, a) spray drying of a liquid enzyme-containing solution, b) production of layered products with an enzyme coated as a layer around a pre-formed inert core particle, e.g. using a fluid bed apparatus, c) absorbing an enzyme onto and/or into the surface of a pre-formed core, d) extrusion of an enzyme-containing paste, e) suspending an enzyme-containing powder in molten wax and atomization to result in prilled products, f) mixer granulation by adding an enzyme-containing liquid to a dry powder composition of granulation components, g) size reduction of enzyme-containing cores by milling or crushing of larger particles, pellets, etc., and h) fluid bed granulation. The enzyme-containing cores may be dried, e.g. using a fluid bed drier or other known methods for drying granules in the feed or enzyme industry, to result in a water content of typically 0.1-10% w/w water.

The enzyme-containing cores are optionally provided with a coating to improve storage stability and/or to reduce dust formation. One type of coating that is often used for enzyme granulates for detergents is a salt coating, typically an inorganic salt coating, which may e.g. be applied as a solution of the salt using a fluid bed. Other coating materials that may be used are, for example, polyethylene glycol (PEG), methyl hydroxy-propyl cellulose (MHPC) and polyvinyl alcohol (PVA). The granules may contain more than one coating, for example a salt coating followed by an additional coating of a material such as PEG, MHPC or PVA.

For further information on enzyme granules and production thereof, see WO 2013/007594 as well as e.g. WO 2009/092699, EP 1705241, EP 1382668, WO 2007/001262, U.S. Pat. No. 6,472,364, WO 2004/074419 and WO 2009/102854.

Uses

The present invention is also directed to methods for using the detergent compositions in laundering of textiles and fabrics, such as household laundry washing and industrial laundry washing.

The invention further relates to the use of the detergent compositions in a cleaning process such as laundering and/or hard surface cleaning such as dishwashing.

A detergent composition of the present invention may be formulated, for example, as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

The cleaning process or the textile care process may for example be a laundry process, a dishwashing process or cleaning of hard surfaces such as bathroom tiles, floors, table tops, drains, sinks and washbasins. Laundry processes can for example be household laundering, but may also be industrial laundering. Furthermore, the invention relates to a process for laundering of fabrics and/or garments, where the process comprises treating fabrics with a washing solution containing a detergent composition of the invention. The cleaning process or a textile care process can for example be carried out in a machine washing or manually. The washing solution can for example be an aqueous washing solution containing a detergent composition.

The invention further concerns the use of the detergent compositions in a proteinaceous stain removing process. The proteinaceous stains may be stains such as food stains, e.g., baby food, cocoa, egg or milk, or other stains such as sebum, blood, ink or grass, or a combination hereof.

In another aspect, the invention relates to a detergent composition comprising 5-100 g of a powder detergent comprising a protease and at least one detergent component, as well as use thereof in a cleaning process, e.g. for laundry or dishwashing, wherein the composition has a pH of not more than about 9 when 15 g of the detergent is dissolved in 15 l of deionized water at 20° C., corresponding to a 1 g/l solution. In this aspect, the composition may e.g. comprise 8-80 g, such as 10-60 g of the powder detergent. In one embodiment, the detergent composition of this aspect is a compact composition, for example in the form of a highly compact powder or a tab, comprising e.g. 10-50 g, such as 10-40 g, such as 10-30 g or 10-20 g, of the powder detergent.

The pH of the composition of this aspect is as outlined further above, i.e. a) from about 7.0 to not more than about 9.0, for example from about 7.2 to about 8.9, such as from about 7.4 to about 8.8, such as from about 7.6 to about 8.7, such as from about 7.8 to about 8.6; b) from about 7.0 to about 8.2, such as from about 7.2 to about 8.0; or c) from about 7.8 to about 8.8, such as from about 8.0 to about 8.6. In this aspect as well, while pH is generally determined with 15 g of the detergent dissolved in 15 l of deionized water (1 g/L), it is contemplated that for unit dosage forms, e.g. tabs, pH may be determined by dissolving one unit, e.g. one tab, in 15 l of deionized water at 20° C., and measuring the pH of this solution.

The composition of this aspect preferably has a conductivity of not more than about 4.0 mS/cm when 15 g of the detergent is dissolved in 15 l of deionized water at 20° C. (i.e. 1 g/l), such as not more than about 3.9 mS/cm, such as not more than about 3.8 mS/cm, such as not more than about 3.7 mS/cm, such as not more than about 3.6 mS/cm, such as not more than about 3.5 mS/cm, such as not more than about 3.4 mS/cm, such as not more than about 3.3 mS/cm, such as not more than about 3.2 mS/cm, such as not more than about 3.1 mS/cm, such as not more than about 3.0 mS/cm, such as not more than about 2.8 mS/cm, such as not more than about 2.6 mS/cm, such as not more than about 2.4 mS/cm, such as not more than about 2.2 mS/cm, or not more than about 2.0 mS/cm. For unit dosage forms, e.g. tabs, conductivity may be determined by dissolving one unit, e.g. one tab, in 15 l of deionized water at 20° C., and measuring the conductivity of this solution.

In another embodiment, the invention relates to the use of a powder detergent composition comprising a protease and at least one detergent component, wherein the composition has a pH of not more than about 9 and a conductivity of not more than about 4.0 mS/cm, wherein pH and conductivity are determined in a 5 g/l solution of the composition in deionized water at 20° C., for providing an improved wash performance compared to a reference composition having a conductivity of 4.2 mS/cm, and preferably compared to a reference composition having a conductivity of 4.5 mS/cm, determined in a 5 g/l solution in deionized water at 20° C. In this embodiment, the reference composition is one that differs from the composition of the invention by having the indicated higher conductivity, but which otherwise is substantially similar to the composition of the invention e.g. in terms of pH.

This aspect further relates to a method of cleaning, especially for cleaning fabrics or textiles, or for dishwashing, comprising contacting fabrics/textiles or dishes with the detergent composition of this aspect under conditions suitable for cleaning the fabrics/textiles or dishes.

The protease in the composition according to this aspect, and for use thereof and a method of cleaning, may be any of the proteases described further above.

A further aspect of the invention relates to use of the proteases described herein in a moderate pH powder detergent composition.

One embodiment of this aspect relates to the use of a protease in a powder detergent composition, wherein the composition has a pH of not more than about 9, wherein pH is determined in a 5 g/l solution of the composition in deionized water at 20° C., and wherein the protease is selected from the group consisting of:

a) a variant of the polypeptide of SEQ ID NO: 1 comprising one of the following sets of mutations, wherein position numbers correspond to positions of the polypeptide of SEQ ID NO: 2:
S99AD;
S99D+S101E+S103A+V104I+G160S;
S3T+V4I+S99D+S101E+S103A+V104I+G160S+V205I;
S3T+V4I+S99D+S101R+S103A+V104I+G160S+V199M+V205I+L217D;
Y167A+R170S+A194P;
S99SE;
S87N+S101G+V104N;
S9E+N43R+N76D+V205I+Q206L+Y209W+S259D+N261W+L262E; or
S99D+S101E+S103A+V104I+S156D+G160S+L262E;

b) the polypeptide of SEQ ID NO: 2 or a variant thereof comprising one of the following sets of mutations, wherein position numbers correspond to positions of the polypeptide of SEQ ID NO: 2:
Y217L;
S24G+S53G+S78N+S101N+G128S+Y217Q; or
S24G+S53G+S78N+S101N+G128A+Y217Q; and

c) the polypeptide of SEQ ID NO: 3 or a variant thereof comprising the mutations S27K+N109K+S111E+S171E+S173P+G174K+S175P+F180Y+G182A+L184F+Q198E+N199K+T297P, wherein position numbers correspond to positions of the polypeptide of SEQ ID NO: 3.

In another embodiment of this aspect the invention relates to the use of a protease in a powder detergent composition, wherein the composition has a pH of not more than about 9, wherein pH is determined in a 5 g/l solution of the composition in deionized water at 20° C., and where the composition further has a conductivity of not more than about 4.0 mS/cm, wherein conductivity is determined in a 5 g/l solution of the composition in deionized water at 20° C., and wherein the protease is a variant of the polypeptide of SEQ ID NO: 1 comprising one of the following sets of mutations, wherein position numbers correspond to positions of the polypeptide of SEQ ID NO: 2:
S99AD;
S99D+S101E+S103A+V104I+G160S;
S3T+V4I+S99D+S101E+S103A+V104I+G160S+V205I;
S3T+V4I+S99D+S101R+S103A+V104I+G160S+V199M+V205I+L217D;
Y167A+R170S+A194P;
S99SE;
S87N+S101G+V104N; or
S9E+N43R+N76D+V205I+Q206L+Y209W+S259D+N261W+L262E.

In the above embodiments relating to use of the proteases described herein in a moderate pH powder detergent composition, it will be apparent that the proteases as well as the pH values and conductivity values may suitably be selected from any of those described in more detail elsewhere herein.

Washing Method

The present invention provides a method of cleaning, especially for cleaning fabrics or textiles, or for dishwashing, with a detergent composition of the invention comprising a protease.

The method of cleaning comprises contacting an object with a detergent composition comprising a protease variant under conditions suitable for cleaning the object. In a preferred embodiment the detergent composition is used in a laundry or dish wash process.

Another embodiment relates to a method for removing stains from fabrics or textiles, which comprises contacting the fabric or textile with a composition of the invention under conditions suitable for cleaning the object.

Another embodiment relates to a method for removing stains from dishware, which comprises contacting the dishware with a composition of the invention under conditions suitable for cleaning the object.

The compositions may be employed at concentrations from about 100 ppm, preferably 500 ppm to about 15,000 ppm in solution. The water temperatures typically range from about 5° C. to about 95° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

The enzyme(s) of the detergent composition of the invention may be stabilized using conventional stabilizing agents and protease inhibitors, e.g., a polyol such as propylene glycol or glycerol, a sugar or sugar alcohol, different salts such as NaCl; KCl; lactic acid, formic acid, boric acid, or a boric acid derivative, e.g., an aromatic borate ester, or a phenyl boronic acid derivative such as 4-formylphenyl boronic acid, or a peptide aldehyde such as di-, tri- or tetrapeptide aldehydes or aldehyde analogues (either of the form B1-B0-R wherein, R is H, CH3, CX3, CHX2, or CH2X (X=halogen), B0 is a single amino acid residue (preferably with an optionally substituted aliphatic or aromatic side chain); and B1 consists of one or more amino acid residues (preferably one, two or three), optionally comprising an N-terminal protection group, or as described in WO 2009/118375, WO 98/13459) or a protease inhibitor of the protein type such as RASI, BASI, WASI (bifunctional alpha-amylase/subtilisin inhibitors of rice, barley and wheat) or C12 or SSI. The composition may be formulated as described in, e.g., WO 92/19709, WO 92/19708 and U.S. Pat. No. 6,472,364. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), Tin (II), cobalt (II), copper (II), Nickel (II), and oxovanadium (IV)).

The present invention is further described by the following examples that should not be construed as limiting the scope of the invention.

### EXAMPLES

### Materials and Methods

### Preparation and Purification of Polypeptides

Mutation and introduction of expression cassettes into Bacillus subtilis was performed by standard methods known in the art. All DNA manipulations were performed by PCR (e.g. as described by Sambrook et al., 2001, supra) using standard methods known to the skilled person.

Recombinant B. subtilis constructs encoding subtilase polypeptides were inoculated into and cultivated in a complex medium (TBgly) under antibiotic selection for 24h at 37° C. Shake flasks containing a rich media (PS-1: 100 g/L Sucrose (Danisco cat.no. 109-0429), 40 g/L crust soy (soy bean flour), 10 g/L Na₂HPO₄.12H₂O (Merck cat.no. 106579), 0.1 ml/L Dowfax63N10 (Dow) were inoculated in a ratio of 1:100 with the overnight culture. Shake flask cultivation was performed for 4 days at 30° C. shaking at 270 rpm.

Purification of culture supernatants was performed as follows: The culture broth is centrifuged at 26000×g for 20 minutes and the supernatant is carefully decanted from the precipitate. The supernatant is filtered through a Nalgene 0.2 μm filtration unit in order to remove the remains of the host cells. The pH in the 0.2 μm filtrate is adjusted to pH 8 with 3 M Tris base and the pH-adjusted filtrate is applied to a MEP Hypercel column (Pall Corporation) equilibrated in 20 mM Tris/HCl, 1 mM CaCl₂), pH 8.0. After washing the column with the equilibration buffer, the column is step-eluted with 20 mM CH₃COOH/NaOH, 1 mM CaCl₂), pH 4.5. Fractions from the column are analyzed for protease activity using the Suc-AAPF-pNA assay at pH 9 and peak fractions are pooled. The pH of the pool from the MEP Hypercel column is adjusted to pH 6 with 20% (v/v) CH3COOH or 3 M Tris base and the pH-adjusted pool is diluted with deionized water to the same conductivity as 20 mM MES/NaOH, 2 mM CaCl₂), pH 6.0. The diluted pool is applied to an SP-Sepharose® Fast Flow column (GE Healthcare) equilibrated in 20 mM MES/NaOH, 2 mM CaCl₂), pH 6.0. After washing the column with the equilibration buffer, the protease variant is eluted with a linear NaCl gradient (0→0.5 M) in the same buffer over five column volumes. Fractions from the column are analyzed for protease activity using the Suc-AAPF-pNA assay at pH 9 and active fractions are analyzed by SDS-PAGE. Fractions in which only one band is observed on the Coomassie stained SDS-PAGE gel are pooled as the purified preparation and used for further experiments.

### Automatic Mechanical Stress Assay (AMSA) for Laundry

In order to assess the wash performance in laundry, washing experiments were performed using the Automatic Mechanical Stress Assay (AMSA). With the AMSA, the wash performance of a large quantity of small volume enzyme-detergent solutions can be examined. The AMSA plate has a number of slots for test solutions and a lid firmly squeezing the laundry sample, the textile to be washed against all the slot openings. During the washing time, the plate, test solutions, textile and lid are vigorously shaken to bring the test solution into contact with the textile and apply mechanical stress in a regular, periodic oscillating manner. For further description see WO02/42740, especially the paragraph “Special method embodiments” at page 23-24.

The laundry experiments were conducted under the experimental conditions specified below:

| | |
|---|---|
| Detergent dosage | 2.5 g/L (Laundry Powder Model Detergent 1) |
| | 5 g/L (Laundry Powder Model Detergent 2) |
| | 5.3 g/L (Laundry Powder Model Detergent 3) |
| | 5.25 g/L (Laundry Powder Model Detergent 4) |
| Enzyme dosage | 2.5-5-10-30 nM |
| Test solution volume | 160 microliters (140 microliters detergent and 20 |
| | microliters enzyme per slot) |
| pH | As is, measured to be: |
| | Laundry Powder Model Detergent 1: 8.2 |
| | Laundry Powder Model Detergent 2: 8.5 |
| | Laundry Powder Model Detergent 3: 8.6 |
| | Laundry Powder Model Detergent 4: 10.2 |
| Wash time | 20 minutes |
| Temperature | 20° C. |
| Water hardness | 15° dH |

Model detergents, proteases and test materials were as follows:

| | |
|---|---|
| Laundry Powder Model | Sodium citrate dihydrate 32.3% |
| Detergent 1 | Sodium-LAS 24.2% |
| | Sodium lauryl sulfate 32.2% |
| | Neodol 25-7 (alcohol ethoxylate) 6.4% |
| | Sodium sulfate 4.9% |
| Laundry Powder Model | Zeolite 43% |
| Detergent 2 | Sodium hydrogen carbonate 24% |
| | Sodium-LAS 18% |
| | Sodium lauryl sulfate 9.5% |
| | Neodol 25-7 (alcohol ethoxylate) 6% |
| Laundry Powder Model | Zeolite 19% |
| Detergent 3 | Sodium hydrogen carbonate 30% |
| | Sodium-LAS 15% |
| | Sodium sulfate 19% |
| | Sodium citrate 10% |
| | Alcohol ethylate 6% |
| | Soap 1% |
| Laundry Powder Model | LAS, sodium salt 11% |
| Detergent 4 | AS, sodium salt 1.8% |
| | Soap, sodium salt 2% |
| | AEO 3% |
| | Soda ash 15% |
| | Hydrous sodium silicate 3% |
| | Zeolite A 20% |
| | HEDP-Na4 0.13% |
| | Sodium citrate 2% |
| | PCA, copoly(acrylic acid/maleic acid), |
| | sodium salt 1.5% |
| | SRP 0.5% |
| | Sodium sulfate 39% |
| | Foam regulator 1% |
| Test Material | EMPA117EH |

The following proteases were tested:

| Protease | |
|---|---|
| number | SEQ ID + mutations |
| Savinase ® | SEQ ID NO: 1 |
| (reference) | |
| 1 | SEQ ID NO: 1 + S99AD |
| 2 | SEQ ID NO: 1 + S87N + S101G + V104N |
| 3 | SEQ ID NO: 1 + S3T + V4I + S99D + S101R + S103A + |
| | V104I + G160S + V199M + V205I + L217D |
| 4 | SEQ ID NO: 3 + S27K + N109K + S111E + S171E + |
| | S173P + G174K + S175P + F180Y + G182A + L184F + |
| | Q198E + N199K + T297P |
| 5 | SEQ ID NO: 1 + Y167A + R170S + A194P |
| 6 | SEQ ID NO: 1 + S99SE |
| 7 | SEQ ID NO: 2 |
| 8 | SEQ ID NO: 2 + Y217L |
| 9 | SEQ ID NO: 1 + S3T + V4I + S99D + S101E + S103A + |
| | V104I + G160S + V205I |
| 10 | SEQ ID NO: 1 + S99D + S101E + S103A + V104I + |
| | G160S |
| 11 | SEQ ID NO: 1 + S9E + N43R + N76D + V205I + |
| | Q206L + Y209W + S259D + N261W + L262E |
| 12 | SEQ ID NO: 3 |
| 13 | SEQ ID NO: 1 + S99D + S101E + S103A + V104I + |
| | S156D + G160S + L262E |
| 14 | SEQ ID NO: 2 + S24G + S53G + S78N + S101N + |
| | G128S + Y217Q |
| 15 | SEQ ID NO: 2 + S24G + S53G + S78N + S101N + |
| | G128A + Y217Q |

Test materials were obtained from EMPA Testmaterials AG, Mövenstrasse 12, CH-9015 St. Gallen, Switzerland.

Water hardness was adjusted to 15° dH by addition of CaCl₂), MgCl₂, and NaHCO₃ (Ca²⁺:Mg²⁺:NaHCO₃=4:1:7.5) to the test system. After washing, the textiles were flushed in tap water and dried.

The wash performance was measured as the brightness of the colour of the washed textile. Brightness can also be expressed as the intensity of the light reflected from the sample when illuminated with white light. When the sample is stained, the intensity of the reflected light is lower than that of a clean sample. Expressed another way, a cleaner sample will reflect more light and will have a higher intensity. Therefore, the intensity of the reflected light can be used to measure wash performance.

Color measurements are made with a professional flatbed scanner (Kodak iQsmart, Kodak, Midtager 29, DK-2605 Brøndby, Denmark), which is used to capture an image of the washed textile.

To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}.

### Example 1

### Wash Performance of a Protease in Different Model Detergents

The wash performance of protease number 1 (SEQ ID NO: 1+S99AD) was investigated in different model detergents using the AMSA method described above. The determined delta intensity values at 20° C. are shown in Table 1.

The pH values indicated for the powder model detergents were measured in a solution of the detergent compositions in water with a hardness of 15° dH at 20° C. using the dosages indicated in the Materials and Methods section above, i.e. for Powder Model Detergents 2, 3 and 4 approximately 5 g/l (between 5 and 5.3 g/l) and for Powder Model Detergent 1 2.5 g/l.

TABLE 1
Determined delta intensity values of protease number
1 relative to detergent without protease at 20°
C. in different laundry model detergents

| Detergent | 2.5 nM | 5 nM | 10 nM | 30 nM |
|---|---|---|---|---|
| Laundry Powder Model Detergent 1 | 36.2 | 41.4 | 46.2 | 55.0 |
| (pH 8.2) | | | | |
| Laundry Powder Model Detergent 2 | 38.8 | 45.0 | 53.4 | 64.2 |
| (pH 8.5) | | | | |
| Laundry Powder Model Detergent 3 | 25.3 | 36.4 | 47.6 | 58.6 |
| (pH 8.6) | | | | |
| Laundry Powder Model Detergent 4 | 12.8 | 28.7 | 36.6 | 53.5 |
| (pH 10.2) | | | | |

From Table 1 it is clear that protease number 1 shows good wash performance in the low pH powder detergents Laundry Powder Model Detergent 1, 2 and 3, whereas a significantly lower performance is detectable in the high pH powder detergent Laundry Powder Model Detergent 4, especially at lower enzyme dosages. This is surprising, as protease number 1 is known to be a protease with a high pl that shows superior wash performance in high pH dish wash detergents, and which was therefore not expected to perform well in a lower pH powder detergent for laundry.

### Example 2

### Relative Wash Performance of Different Proteases in Different Model Detergents

The relative wash performance of proteases 1-15 with the sequences and mutations shown above was investigated in four different model powder detergents using the AMSA method. Performance was compared to that of the reference protease Savinase®, the wash performance of which was set to 1. Table 2 below shows the calculated relative wash performance of the different proteases in the four model detergents determined at 20° C. compared to Savinase®.

Table 2 also shows, in addition to the pH values of the detergent solutions, the measured conductivity at the dosages indicated above in the Materials and Methods section. Referring to the detergent dosages indicated above in the Materials and Methods section, it is apparent that Laundry Powder Model Detergent 1 was dosed differently from a “standard” dosage of 5 g/L. This is because the different model detergents have been dosed herein for purposes of determining wash performance in amounts that approximate typical dosages recommended by manufacturers for the detergent types in question in the relevant markets. Conductivity has similarly been determined using 15° dH water with the individual detergent dosages rather than in deionized water.

TABLE 2
Relative wash performance of different proteases in model
powder detergents compared to Savinase ®

| | Detergent: | | | |
|---|---|---|---|---|
| | Model | Model | Model | Model |
| | Detergent 1 | Detergent 2 | Detergent 3 | Detergent 4 |
| | pH: | | | |
| | 8.2 | 8.5 | 8.6 | 10.2 |
| | Conductivity: (mS/cm) | | | |
| | 1.4 | 2.5 | 4.2 | 4.5 |
| Protease | Relative wash performance | | | |
| Savinase ® | 1.00 | 1.00 | 1.00 | 1.00 |
| (reference) | | | | |
| 1 | 2.01 | 2.37 | 2.19 | 0.83 |
| 2 | 1.40 | 1.73 | 2.13 | 1.20 |
| 3 | 1.99 | 2.59 | 3.11 | 1.51 |
| 4 | 2.43 | 2.33 | 0.50 | 0.03 |
| 5 | 2.14 | 2.48 | 2.28 | 0.90 |
| 6 | 2.16 | 2.56 | 1.67 | 0.70 |
| 7 | 1.53 | 1.64 | 0.68 | 0.30 |
| 8 | 1.58 | 1.70 | 0.67 | 0.18 |
| 9 | 2.35 | 2.54 | 1.98 | 0.68 |
| 10 | 2.22 | 2.74 | 2.37 | 0.69 |
| 11 | 1.27 | 1.38 | 1.19 | 0.81 |
| 12 | 2.19 | 2.04 | 0.43 | 0.08 |
| 13 | 2.56 | 2.43 | 0.68 | 0.17 |
| 14 | 2.50 | 2.31 | 0.47 | 0.01 |
| 15 | 2.41 | 2.37 | 0.75 | 0.02 |

The results in Table 2 show that most of the tested proteases have a poor performance in the high pH Model Detergent 4 compared to performance of the reference protease Savinase®. On the other hand, all the tested proteases show improved relative performance in the low pH Model Detergents 1 and 2, while the majority also show improved performance in the low pH Model Detergent 3. It is apparent that some of the tested proteases (4, 7, 8, 12, 13, 14 and 15) have a poor relative performance in the low pH Model Detergent 3, while still showing a substantially improved relative performance in the other low pH Model Detergents 1 and 2.

It is believed that this is related to the fact that the Model Detergent 3 has a relatively high conductivity of 4.2 mS/cm. In contrast, the other two low pH Model Detergents 1 and 2 have much lower conductivities of 1.4 and 2.5 mS/cm, respectively. Thus, for some proteases it is sufficient that the detergent composition has a relatively low pH to obtain improved relative wash performance, while for others not only a low pH but also a low conductivity appears to be required. In any event, the combination of a relatively low pH and a relatively low conductivity in Model Detergents 1 and 2 resulted in an improved relative performance of all of proteases 1-15.

1. A powder detergent composition comprising a protease and at least one detergent component, wherein the composition has a pH of not more than about 9, wherein pH is determined in a 5 g/l solution of the composition in deionized water at 20° C.

2. The composition of claim 1, wherein the composition has a conductivity of not more than about 4.0 mS/cm, wherein conductivity is determined in a 5 g/l solution of the composition in deionized water at 20° C.

3. The composition of claim 1, wherein the composition has a pH determined as defined in claim 1 of a) from about 7.0 to not more than about 9.0, for example from about 7.2 to about 8.9, such as from about 7.4 to about 8.8, such as from about 7.6 to about 8.7, such as from about 7.8 to about 8.6; b) from about 7.0 to about 8.2, such as from about 7.2 to about 8.0; or c) from about 7.8 to about 8.8, such as from about 8.0 to about 8.6.

4. The composition of claim 2, wherein the composition has a conductivity of not more than about 3.9 mS/cm, such as not more than about 3.8 mS/cm, such as not more than about 3.7 mS/cm, such as not more than about 3.6 mS/cm, such as not more than about 3.5 mS/cm, such as not more than about 3.4 mS/cm, such as not more than about 3.3 mS/cm, such as not more than about 3.2 mS/cm, such as not more than about 3.1 mS/cm, such as not more than about 3.0 mS/cm, such as not more than about 2.8 mS/cm, such as not more than about 2.6 mS/cm, such as not more than about 2.4 mS/cm, such as not more than about 2.2 mS/cm, or not more than about 2.0 mS/cm.

5. The composition of claim 1, wherein the protease is selected from the group consisting of:
a) a variant of the polypeptide of SEQ ID NO: 1, wherein the variant has protease activity and at least 70%, at least 75%, at least 80%, at least 85%, at least 90% or at least 95% but less than 100% sequence identity to SEQ ID NO: 1;
b) the polypeptide of SEQ ID NO: 2 or a variant thereof, wherein the variant has protease activity and at least 70%, at least 75%, at least 80%, at least 85%, at least 90% or at least 95% but less than 100% sequence identity to SEQ ID NO: 2; and
c) the polypeptide of SEQ ID NO: 3 or a variant thereof, wherein the variant has protease activity and at least 70%, at least 75%, at least 80%, at least 85%, at least 90% or at least 95% but less than 100% sequence identity to SEQ ID NO: 3.

6. The composition of claim 1, wherein the protease is a variant of the polypeptide of SEQ ID NO: 1 comprising one of the following sets of mutations, wherein position numbers correspond to positions of the polypeptide of SEQ ID NO: 2:
S99AD;
S99D+S101E+S103A+V104I+G160S;
S3T+V4I+S99D+S101E+S103A+V104I+G160S+V205I;
S3T+V4I+S99D+S101R+S103A+V104I+G160S+V199M+V205I+L217D;
Y167A+R170S+A194P;
S99SE;
S87N+S101G+V104N;
S9E+N43R+N76D+V205I+Q206L+Y209W+S259D+N261W+L262E; or
S99D+S101E+S103A+V104I+S156D+G160S+L262E;
wherein the protease has at least 80%, at least 85%, at least 90% or at least 95% sequence identity to SEQ ID NO: 1 and comprises one of said sets of mutations, or wherein the protease comprises or consists of the polypeptide of SEQ ID NO: 1 with one of said sets of mutations.

7. The composition of claim 1, wherein the protease is
a) the polypeptide of SEQ ID NO: 2; or
b) a variant of SEQ ID NO: 2 comprising one of the following sets of mutations:
Y217L;
S24G+S53G+S78N+S101N+G128S+Y217Q; and
S24G+S53G+S78N+S101N+G128A+Y217Q;
wherein the protease variant has at least 80%, at least 85%, at least 90% or at least 95% sequence identity to SEQ ID NO: 2 and comprises one of said sets of mutations, or wherein the protease variant comprises or consists of the polypeptide of SEQ ID NO: 2 with one of said sets of mutations.

8. The composition of claim 1, wherein the protease is the polypeptide of SEQ ID NO: 3 or a variant thereof having at least 80%, at least 85%, at least 90% or at least 95% sequence identity to SEQ ID NO: 3, for example a variant comprising one or more mutations selected from the group consisting of S27K, N109K, S111E, S171E, S173P, G174K, S175P, F180Y, G182A, L184F, Q198E, N199K and T297P, e.g. a variant of SEQ ID NO: 3 comprising the mutations
S27K+N109K+S111E+S171E+S173P+G174K+S175P+F180Y+G182A+L184F+Q198E+N199K+T297P.

9. (canceled)

10. A method of cleaning, especially for cleaning fabrics or textiles, or for dishwashing, comprising contacting fabrics/textiles or dishes with a detergent composition according to any of claims 1-8 under conditions suitable for cleaning the fabrics/textiles or dishes.

11. A method of cleaning comprising contacting a detergent composition comprising 5-100 g of a powder detergent comprising a protease and at least one detergent component with an item to be cleaned wherein the composition has a pH of not more than about 9 when 15 g of the detergent is dissolved in 15 l of deionized water at 20° C.

12. The method of claim 11, wherein the pH is a) from about 7.0 to not more than about 9.0, for example from about 7.2 to about 8.9, such as from about 7.4 to about 8.8, such as from about 7.6 to about 8.7, such as from about 7.8 to about 8.6; b) from about 7.0 to about 8.2, such as from about 7.2 to about 8.0; or c) from about 7.8 to about 8.8, such as from about 8.0 to about 8.6.

13. The method of claim 11, wherein the composition has a conductivity of not more than about 4.0 mS/cm when 15 g of the detergent is dissolved in 15 l of deionized water at 20° C., such as not more than about 3.9 mS/cm, such as not more than about 3.8 mS/cm, such as not more than about 3.7 mS/cm, such as not more than about 3.6 mS/cm, such as not more than about 3.5 mS/cm, such as not more than about 3.4 mS/cm, such as not more than about 3.3 mS/cm, such as not more than about 3.2 mS/cm, such as not more than about 3.1 mS/cm, such as not more than about 3.0 mS/cm, such as not more than about 2.8 mS/cm, such as not more than about 2.6 mS/cm, such as not more than about 2.4 mS/cm, such as not more than about 2.2 mS/cm, or not more than about 2.0 mS/cm.

14. A detergent composition comprising 5-100 g of a powder detergent comprising a protease and at least one detergent component, wherein the composition has a pH of not more than about 9 when 15 g of the detergent is dissolved in 15 l of deionized water at 20° C.

15. The composition of claim 14, wherein the pH is a) from about 7.0 to not more than about 9.0, for example from about 7.2 to about 8.9, such as from about 7.4 to about 8.8, such as from about 7.6 to about 8.7, such as from about 7.8 to about 8.6; b) from about 7.0 to about 8.2, such as from about 7.2 to about 8.0; or c) from about 7.8 to about 8.8, such as from about 8.0 to about 8.6.

16. The composition of claim 14, wherein the composition has a conductivity of not more than about 4.0 mS/cm when 15 g of the detergent is dissolved in 15 l of deionized water at 20° C., such as not more than about 3.9 mS/cm, such as not more than about 3.8 mS/cm, such as not more than about 3.7 mS/cm, such as not more than about 3.6 mS/cm, such as not more than about 3.5 mS/cm, such as not more than about 3.4 mS/cm, such as not more than about 3.3 mS/cm, such as not more than about 3.2 mS/cm, such as not more than about 3.1 mS/cm, such as not more than about 3.0 mS/cm, such as not more than about 2.8 mS/cm, such as not more than about 2.6 mS/cm, such as not more than about 2.4 mS/cm, such as not more than about 2.2 mS/cm, or not more than about 2.0 mS/cm.

17. (canceled)

18. A method of cleaning comprising contacting a powder detergent composition comprising a protease and at least one detergent component, wherein the composition has a pH of not more than about 9 and a conductivity of not more than about 4.0 mS/cm, wherein pH and conductivity are determined in a 5 g/l solution of the composition in deionized water at 20° C., with an item to be cleaned for providing an improved wash performance compared to a reference composition having a conductivity of 4.2 mS/cm, and preferably compared to a reference composition having a conductivity of 4.5 mS/cm, determined in a 5 g/l solution in deionized water at 20° C.

19. A method of washing an item comprising contacting the item to be washed with a protease in a powder detergent composition, wherein the composition has a pH of not more than about 9, wherein pH is determined in a 5 g/l solution of the composition in deionized water at 20° C., and wherein the protease is selected from the group consisting of:
a) a variant of the polypeptide of SEQ ID NO: 1 comprising one of the following sets of mutations, wherein position numbers correspond to positions of the polypeptide of SEQ ID NO: 2:
S99AD;
S99D+S101E+S103A+V104I+G160S;
S3T+V4I+S99D+S101E+S103A+V104I+G160S+V205I;
S3T+V4I+S99D+S101R+S103A+V104I+G160S+V199M+V205I+L217D;
Y167A+R170S+A194P;
S99SE;
S87N+S101G+V104N;
S9E+N43R+N76D+V205I+Q206L+Y209W+S259D+N261W+L262E; or
S99D+S101E+S103A+V104I+S156D+G160S+L262E;
b) the polypeptide of SEQ ID NO: 2 or a variant thereof comprising one of the following sets of mutations, wherein position numbers correspond to positions of the polypeptide of SEQ ID NO: 2:
Y217L;
S24G+S53G+S78N+S101N+G128S+Y217Q; or
S24G+S53G+S78N+S101N+G128A+Y217Q; and
c) the polypeptide of SEQ ID NO: 3 or a variant thereof comprising the mutations S27K+N109K+S111E+S171E+S173P+G174K+S175P+F180Y+G182A+L184F+Q198E+N199K+T297P, wherein position numbers correspond to positions of the polypeptide of SEQ ID NO: 3.

20. The method of claim 19, for providing an improved wash performance compared to a reference composition having a pH of 10, wherein pH is determined in a 5 g/l solution in deionized water at 20° C.

21. A method of washing an item comprising contacting the item to be washed with a protease in a powder detergent composition, wherein the composition has a pH of not more than about 9, wherein pH is determined in a 5 g/l solution of the composition in deionized water at 20° C., and where the composition further has a conductivity of not more than about 4.0 mS/cm, wherein conductivity is determined in a 5 g/l solution of the composition in deionized water at 20° C., and wherein the protease is a variant of the polypeptide of SEQ ID NO: 1 comprising one of the following sets of mutations, wherein position numbers correspond to positions of the polypeptide of SEQ ID NO: 2:
S99AD;
S99D+S101E+S103A+V104I+G160S;
S3T+V4I+S99D+S101E+S103A+V104I+G160S+V205I;
S3T+V4I+S99D+S101R+S103A+V104I+G160S+V199M+V205I+L217D;
Y167A+R170S+A194P;
S99SE;
S87N+S101G+V104N; or
S9E+N43R+N76D+V205I+Q206L+Y209W+S259D+N261W+L262E.

22. The method of claim 21, for providing an improved wash performance compared to a reference composition having a conductivity of 4.2 mS/cm, and preferably compared to a reference composition having a conductivity of 4.5 mS/cm, determined in a 5 g/l solution in deionized water at 20° C.
